(19) United States
(12) Patent Application Publication
(10) Pub. No.: US 2012/0301402 A1
(43) Pub. Date: Nov. 29, 2012

(54) RAP1A AS A MARKER FOR CARDIAC ARRHYTHMIA

(75) Inventors: Maqsood Ahmed Chotani, Columbus, OH (US); John Anthony Bauer, Westerville, OH (US); Mandar Shankar Joshi, Glen Waverly (AU); Selvi Chrysolyte Jeyaraj, Columbus, OH (US)

(73) Assignee: NATIONWIDE CHILDREN'S HOSPITAL, INC., Columbus, OH (US)

(21) Appl. No.: 13/458,079

(22) Filed: Apr. 27, 2012

Related U.S. Application Data
(60) Provisional application No. 61/479,760, filed on Apr. 27, 2011.

(51) Int. Cl. G01N 33/573; A61K 49/00; A61K 31/4709; A61K 31/166; A61K 31/4402; A61P 9/06; A61K 31/4166; A61K 31/138; A61K 31/4458; A61K 31/5415; A61K 31/24; A61K 31/5377; C12Q 1/02; A61K 31/167

(52) U.S. Cl. 424/9.2; 435/7.4; 435/29; 514/305; 514/619; 514/357; 514/626; 514/391; 514/651; 514/331; 514/652; 514/225.2; 514/538; 514/236.2

(57) ABSTRACT

A method for assessing if a subject has or is at risk of developing cardiac arrhythmia is described. The method includes determining the activity of Rap1A protein in a bodily sample of the subject and comparing the activity of Rap1A protein from the bodily sample of the subject to at least one Rap1A control. A decreased level of Rap1A activity in the bodily sample of the subject as compared to the at least one Rap1A control indicates that the subject is at risk of developing or has cardiac arrhythmia. Methods for evaluating the efficacy of treatment of a subject having decreased Rap1A activity with an antiarrhythmic agent are also described.

### CROSS-REFERENCE TO RELATED APPLICATIONS

This application claims priority to U.S. Provisional Patent Application No. 61/479,760, filed Apr. 27, 2011, which is hereby incorporated by reference in its entirety.

### STATEMENT REGARDING FEDERALLY FUNDED RESEARCH

These inventions were made with Government support under Institutional Training Grant T32HL098039-01 (NHLBI), R21-HL088087 (NHLBI), R41AT005193 (NCCAM), and R41CA121664 grants awarded by the National Institutes of Health. The United States government has certain rights in this invention.

### BACKGROUND

The human heart is a muscular organ that provides a continuous blood circulation through the cardiac cycle and is one of the most vital organs in the human body. The heart is divided into four main chambers: the two upper chambers are called the left and right atria and two lower chambers are called the right and left ventricles. There is a thick wall of muscle separating the right side and the left side of the heart called the septum. Normally with each beat the right ventricle pumps the same amount of blood into the lungs that the left ventricle pumps out into the body. Physicians commonly refer to the right atrium and right ventricle together as the right heart and to the left atrium and ventricle as the left heart.

The electric energy that stimulates the heart occurs in the sinoatrial node, which produces a definite potential and then discharges, sending an impulse across the atria. In the atria the electrical signal moves from cell to cell while in the ventricles the signal is carried by specialized tissue called the Purkinje fibers that then transmit the electric charge to the myocardium.

The normal electrical conduction of the heart allows electrical propagation to be transmitted from the sinoatrial (SA) node through both atria and forward to the atrioventricular (AV) node. Normal/baseline physiology allows further propagation from the AV node to the ventricle or Purkinje fibers and respective bundle branches and subdivisions/fascicles. Both the SA and AV nodes stimulate the myocardium. Time ordered stimulation of the myocardium allows efficient contraction of all four chambers of the heart, thereby allowing selective blood perfusion through both the lungs and systemic circulation.

Neurons innervating cardiac musculature bear limited similarities to those of skeletal muscle, as well as having other important differences. Cardiac neurons are uniquely subject to influence by the sympathetic and parasympathetic influence of the autonomic nervous system unlike skeletal muscle. Like a neuron, a given myocardial cell has a negative membrane potential when at rest. Stimulation above a threshold value induces the opening of voltage-gated ion channels with inducted flow of cations into the cell. The positively charged ions entering the cell cause the depolarization characteristic of an action potential. After depolarization, there is a brief repolarization that takes place with the efflux of potassium through fast-acting potassium channels. Like skeletal muscle, depolarization causes the opening of voltage-gated calcium channels, meanwhile potassium channels have closed, and are followed by a titrated release of Ca²⁺ from the t-tubules. This influx of calcium causes calcium-induced calcium release from the sarcoplasmic reticulum, and free Ca²⁺ causes muscle contraction. After a delay, slow-acting potassium channels reopen and the resulting flow of K⁺ out of the cell causes repolarization to the resting state.

The QT interval is a measure of the time between the start of the Q wave and the end of the T wave in the heart's electrical cycle. In general, the QT interval represents electrical depolarization and repolarization of the left and right ventricles. A prolonged QT interval is a biomarker for ventricular twitching or fibrillation and for ventricular tachyarrhythmias like torsades de pointes (TDP, a form of irregular heartbeat that originates from the ventricles) and a risk factor for sudden death.

The QT interval is an important electrocardiogram (ECG) parameter and the identification of ECGs with long QT syndrome is of clinical importance. Considering the required standards for precision, the measurement of QT interval is subjective. This is because the end of the T wave is not always clearly defined and usually merges gradually with the baseline. QT interval can be measured manually by different methods such as the threshold method, in which the end of the T wave is determined by the point at which the component of the T wave merges with the isoelectric baseline or the tangent method, in which the end of the T wave is determined by the intersection of a line extrapolated from the isoelectric baseline and the tangent line, which touches the terminal part of the T wave at the point of maximum downslope.

If the QT interval is abnormally prolonged or shortened, there is a risk of developing ventricular arrhythmias. An abnormal prolonged QT interval could be due to “long QT syndrome,” (LQTS) whereas an abnormal shortened QT interval could be due to “short QT syndrome.” The length of the interval has also been found to associate with numerous conditions, such as, for example, adverse drug reactions and pathological conditions. Many drugs such as haloperidol and methadone can prolong the QT interval. Some anti-arrhythmic drugs, like amiodarone or sotalol work by inducing a pharmacological QT prolongation. Additionally, some second generation antihistamines, such as astemizole, have this effect. Additionally, alcohol in high blood concentrations can prolong the QT interval. Hypothyroidism, a condition of low function of the thyroid gland, can result in QT interval prolongation. A shortened QT can be associated with hypercalcemia.

LQTS is a rare inborn heart condition in which delayed repolarization of the heart following a heartbeat increases the risk of episodes of TDP. These episodes may lead to palpitations, fainting, and sudden death due to ventricular fibrillation. Episodes may be provoked by various stimuli, depending on the subtype of the condition. The condition is so named because of the appearances of the ECG, on which there is prolongation of the QT interval. In some individuals the QT prolongation only occurs after the administration of certain medications.

LQTS can arise from mutation of one of several genes. These mutations tend to prolong the duration of the ventricular action potential (APD), thus lengthening the QT interval. LQTS can be inherited in an autosomal dominant or an autosomal recessive fashion. The autosomal recessive forms of LQTS tend to have a more severe phenotype, with some variants having associated syndactyly (LQT8) or congenital neural deafness (LQT1). A number of specific gene loci have been identified that are associated with LQTS. Genetic testing for LQTS is clinically available and may help to direct appropriate therapies

The most common causes of LQTS are mutations in the genes KCNQ1 (LQT1), KCNH2 (LQT2), and SCN5A (LQT3). The following is a list of all known genes associated with LQTS: alpha subunit of the slow delayed rectifier potassium channel (KvLQT1 or KCNQ1); alpha subunit of the rapid delayed rectifier potassium channel (HERG+MiRP1); alpha subunit of the sodium channel (SCN5A); anchor protein Ankyrin B; beta subunit MinK (or KCNE1) which co-assembles with KvLQT1; beta subunit MiRP1 (or KCNE2) which co-assembles with HERG; potassium channel KCNJ2 (or Kᵢᵣ2.1); alpha subunit of the calcium channel Cav1.2 encoded by the gene CACNA1c; Caveolin 3; SCN4B; AKAP9; SNTA1; and GIRK4. However, the identification of new markers for LQTS and genes associated with LQTS is desirable since the diagnosis of LQTS is not easy. 2.5% of the healthy population have prolonged QT interval, and 10-15% of LQTS patients have a normal QT interval.

With better knowledge of the genetics underlying long QT syndrome, more precise treatments can be developed for administration to subjects in need of such treatments, better methods for assessing if a subject is at risk of developing or has LQTS can be developed, and a more accurate and thorough evaluation of QT liability of drugs can be performed.

### SUMMARY

Cardiac arrhythmias are major health risks and in general few drugs are available to prevent or treat such disease states. Thus far most agents that have been developed as anti-arrhythmia therapies have targeted one or more ion channels involved in normal cardiac myocyte electrical activity, but these often have dangerous side effects or a narrow therapeutic window. Disclosed herein are findings that demonstrate that a protein, Rap1A, which has not been considered relevant in this setting, may be an important regulator of cardiac arrhythmias.

In aspect, a method for assessing if a subject has or is at risk of developing cardiac arrhythmia is described. The method includes determining the activity of Rap1A protein in a bodily sample of the subject; and comparing the activity of Rap1A protein from the bodily sample of the subject to at least one Rap1A control, wherein a decreased level of Rap1A activity in the bodily sample of the subject as compared to the at least one Rap1A control indicates that the subject is at risk of developing or has cardiac arrhythmia. In one embodiment, the cardiac arrhythmia identified by the method is Long QT syndrome. In another embodiment, the method further includes the step of administering a therapeutically effective amount of an antiarrhythmic agent to a subject that has or is at risk of developing cardiac arrhythmia.

In another aspect, a method of evaluating the efficacy of treatment of a subject having decreased Rap1A activity with an antiarrhythmic agent is described. The method includes the steps of: a) identifying a subject having decreased Rap1A activity, b) administering a therapeutically effective amount of an antiarrhythmic agent to the subject, c) determining the level of a symptom of cardiac arrhythmia in the subject; d) comparing the level of the symptom of cardiac arrhythmia to a corresponding predetermined value, and determining the treatment to be efficacious if the level of the symptom of cardiac arrhythmia has decreased in comparison to the predetermined value.

In an additional embodiment, the symptom of cardiac arrhythmia is increased cardiovascular sodium channel activity. In another embodiment, the symptom of cardiac arrhythmia is prolongation of the QT interval. In yet another embodiment, the symptom of cardiac arrhythmia is palpitations.

### BRIEF DESCRIPTION OF THE FIGURES

The accompanying drawings, which are incorporated in and constitute a part of this specification, illustrate some embodiments, and together with the description, serve to explain principles of the embodiments.

FIG. 1 provides a schematic illustrating the biological function of Rap1. Rap1A acts as a molecular on/off switch and cycles between a GTP bound (active state) and a GDP bound (inactive state). Activation of cell surface stimulatory G protein (Gs) coupled β2-adrenoceptors (β-ARs) by the endogenous agonist norepinephrine or by the synthetic catecholamine isoproterenol (ISO) leads to increased intracellular production of the second messenger cyclic AMP by the enzyme adenylyl cyclase and activation of Rap1A by the guanine nucleotide exchange factor EPAC (the exchange protein activated by cAMP). Activated Rap1 mediates biological responses through interactions with effector proteins.

FIG. 2 provides bar graphs showing the long QT interval in Rap1A−/− mice. In vivo cardiovascular status of Rap1A knockout (−/−) mice by electrocardiography (ECG) (n=5/group) in comparison to wild-type (+/+) controls. Recordings were performed on anesthetized mice using six-lead ECG configuration. The QT corrections factors were tested to validate the data. The average RR interval was determined from a 30 second sample. Rate corrected QT interval, QTc, was calculated using the methods of Bazett (QTc=QT/RR^{1/2}) and Fridericia (QTc=QT/RR^{1/3}). QRS interval was also determined. *P<0.05, statistically significant difference.

FIG. 3 provides bar graphs of qRT-PCR results showing altered potassium channel mRNA expression. mRNA from Rap1A−/− and Rap1A+/+ mice (n=4) was isolated from heart tissue, reverse transcribed and subjected to qPCR with primers specific to KCNQ1 and KCNH2 potassium channels. Expression of both channels was decreased in mice lacking Rap1A. *P<0.05, statistically significant difference.

FIG. 4 provides a picture showing the altered expression of DNA methyltransferase Dnmt3a in Rap1A knockout hearts. Heart tissue from Rap1A−/− and control+/+ mice was harvested and protein extracted. Total Dnmt3a protein expression was determined by Western analysis. The housekeeping gene glyceraldehyde-3-phosphate dehydrogenase (GAPDH) was included as a loading control.

FIG. 5 provides graphs showing heart rates following isoproterenol challenge. Heart rates were determined from surface electrocardiogram (ECG) recordings of C57BL/6J male mice, control wild-type (WT) or mice deficient in the Ras-related GTPase Rap1A (Rap1A-knockout (KO) or Rap1A-heterozygous (Het). Recordings were performed in mice under isoflurane anesthesia. Normal body temperature was maintained during data collection. Cardiac output was increased by intraperitoneal injection of the non-selective beta-adrenergic activator isoproterenol, used to simulate elevated sympathetic system drive. (A) Heart rates (wild-type (n=6) and Rap1A-Het (n=5) mice) showing no significant change in Rap1A-Het mice to isoproterenol dose response (0-1 mg/kg), and (B) following isoproterenol challenge examined in Rap1A-KO mice (isoproterenol, 0.4 mg/kg).

FIG. 6 provides graphs and pictures showing cardiovascular function and ultrastructure. (A) The in vivo cardiovascular status of control wild-type (WT) and Rap1A-knockout mice was assessed by non-invasive Echocardiography (Echo). Recordings were performed in mice under isoflurane anesthesia. Normal body temperature was maintained during data collection. Cardiac output was increased by intraperitoneal injection of the beta-adrenergic activator isoproterenol (ISO). Two dimensional (entire left ventricle) and M-mode (shape changes of a single region over time) Echo images were recorded and analyzed by using the Visual Sonics VEVO 2100 Echo imaging system at 30 MHz (n=3 mice/group). Left ventricle performance was assessed by—ejection fraction (measure of the percent of blood leaving the heart each time it contracts), and fractional shortening (change in diameter of left ventricle between contracted and relaxed states). (B) Fine structure of cardiac myocytes from Rap1A-knockout (−/−) and wild-type (+/+) controls was evaluated by transmission electron microscopy, TEM. Tissue samples were cut to 1 mm cubes and processed using standard protocol. Sample blocks were trimmed and 500 nm thick sections were cut and collected onto slides. Slides were stained with Toluidine blue and assessed for areas of interest. Selected blocks were cut at 60 nm on a Leica Ultracut UCT ultramicrotome. Sections were collected onto CuPd grids and stained with uranyl acetate and lead citrate. Grids were viewed on a Hitachi H 7650 TEM. Cardiac myocytes with prominent Z-bands and an abundance of mitochondria are apparent in both knockout and control tissues. Rap1A-knockout cardiac myocytes have no apparent structural abnormalities.

FIG. 7 provides graphs showing calcium (Ca²⁺) handling in isolated cardiac myocytes. Ventricular myocytes were isolated from male C57BL/6 control wild-type (WT) and Rap1A-heterozygous (Rap1a^{+/-}) mice. Whole hearts were cannulated and hung on a Langendorff apparatus and tissue treated for enzymatic digestion for 3-5 minutes. The ventricles were minced and myocytes were dissociated by trituration. Subsequently, the myocytes were filtered, centrifuged, and used for the studies within 6 hours of isolation. Calcium transients were measured by loading isolated myocytes with fluo 4-AM (10 μM) for 30 min, followed by an additional 30 min for intracellular deesterification. Cell fluorescence was measured by epifluorescence. The intracellular calcium concentration [Ca²⁺]ᵢ of single cells was measured with excitation at 480±20 nm and emission at 535±25 nm. Data were expressed as ΔF/F₀, where F is the fluorescence intensity, and F₀ is the intensity at rest. Myocytes were stimulated at 1 Hz. Simultaneous measurement of shortening was also performed using edge detection. Data were expressed as cell shortening (μm). Myocytes were analyzed from 2-3 independent isolations per group. (A) Similar calcium (Ca²⁺) handling and contraction were observed for WT and Rap1a^{+/-} myocytes. (B) Similar beta-adrenergic responses were observed by using the beta agonist isoproterenol 1 μM).

FIG. 8 provides graphs and pictures showing spontaneous calcium transients or after-transients in Rap1A-heterozygous and Rap1A-homozygous isolated cardiac myocytes. After-transients do not reflect a change in altered signaling. (A) Heterozygous (grey bar) and homozygous (clear bar) develop more after-transients during beta-adrenergic stimulation compared to control wild-type (WT, black bar) myocytes. The sarcoplasmic reticulum (SR) calcium (Ca²⁺) load in Rap1A-heterozygous (Rap 1a^{+/-}) myocytes is unaltered, whereas RT₅₀ (time to 50% of the calcium transient decline) following release of calcium load in response to caffeine is increased in Rap1A-heterozygous myocytes. Myocytes were analyzed from 2-3 independent isolations per group. (B) Expression of SR regulating SERCA protein was unaltered (n=4/group, P=NS). (C) No change in expression of calcium release and uptake proteins ryanodine (RYR) and phospholamban (PLB) were observed, and the phosphorylation of these proteins in response to beta-adrenergic stimulation with isoproterenol was unchanged (n=6/group, P=NS).

FIG. 9 provides graphs and pictures showing after-transients and rescue of sodium channels. (A) After-transients in isolated myocytes can be rescued by a non-specific sodium (Na+) channel blocker Mexiletine. In order to examine the mechanism involved in the spontaneous wave, the role of Na⁺ channels (SCN5A/Nav1.5) was examined during isoproterenol treatments. The combination of isoproterenol and Mexiletine rescued the spontaneous calcium waves. The spontaneous waves resumed after washout of Mexiletine. (B) No significant alteration in mRNA or protein levels was observed for membrane channels affecting sodium current (SCN5A and NCX-1), determined by quantitative RT-PCR and Western blot analysis (n=4/group, P=NS).

FIG. 10 provides graphs showing that aftercontractions increase in heterozygous Rap1A myocytes. Further investigation of the spontaneous waves was performed using an “after-contractions” protocol by stimulating for 5 seconds and counting the number of after-contractions that occur 40 seconds after the stimulation is withdrawn. (A) Rap1A-heterozygous myocytes have increased after-contractions compared to control wild-type (WT) myocytes. (B) Inhibition of protein kinase C (PKC) by bisindolylmaleimide I (BIM, 1 μM) increases after-contractions in control wild-type (WT) and Rap1A-heterozygous myocytes. (C) Protein kinase C (PKC) activation by phorbol myristate acetate (PMA, 150 nM) leads to a decrease in after-contractions in Rap1A-heterozygous myocytes. (D) Summary data of WT/Rap1A-heterozygous+/− BIM (Inhibitor) and PMA (Activator). Both drugs were incubated in isolated ventricular myocytes before measurements were taken to activate or inhibit PKC.

FIG. 11 shows the after-transients produced by patch-clamping isolated Rap1A-heterozygous cardiac myocytes. The after-transients appeared under period stimulation (1 Hz) conditions.

FIG. 12 provides a schematic of Rap1A function in cardiac myocytes. According to this model stimulation of beta-adrenergic receptors activates Rap1A leading to protein kinase C (PKC) mediated phosphorylation of amino acid serine at position 1503 in the sodium channel SCNA5A, leading to inactivation of the channel. In the absence of Rap1A signaling (Rap1A-heterozygous or homozygous myocytes), SCN5A has increased function. Increased channel function is known to be associated with human mutations ΔKPD, Y1795C, and Y1795H. According to this model beta-adrenergic activation of Rap1A-PKC pathway will phosphorylate Serine1503 and inhibit channel activity. This model explains the anti-arrhythmic effect of beta-adrenergic activation in known human carriers of long QT-3 (LQT3) mutations.

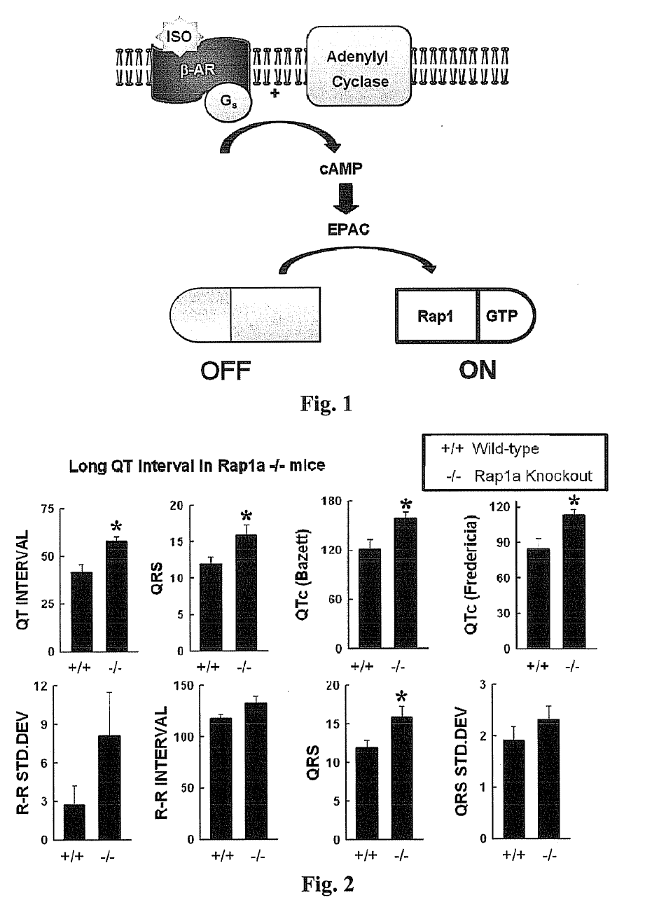

### DETAILED DESCRIPTION

The present embodiments will now be described by reference to some more detailed embodiments. However, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### DEFINITIONS

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sample” includes a plurality of such samples.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

A method for assessing if a subject has or is at risk of developing cardiac arrhythmia is described. The method includes determining the activity of Rap1A protein in a bodily sample of the subject and comparing the activity of Rap1A protein from the bodily sample of the subject to at least one Rap1A control. A decreased level of Rap1A activity in the bodily sample of the subject as compared to the at least one Rap1A control indicates that the subject is at risk of developing or has cardiac arrhythmia. Methods for evaluating the efficacy of treatment of a subject having decreased Rap1A activity with an antiarrhythmic agent are also described.

### The Rap1A Protein

Ras-related protein Rap1A is a protein that in humans is encoded by the RAP1A gene. The product of this gene belongs to the family of Ras-related proteins. These proteins share approximately 50% amino acid identity with the classical RAS proteins and have numerous structural features in common. The most striking difference between RAP proteins and RAS proteins resides in their 61st amino acid: glutamine in RAS is replaced by threonine in RAP proteins. The product of this gene counteracts the mitogenic function of RAS because it can interact with RAS GAPs and RAF in a competitive manner. Two transcript variants encoding the same protein have thus far been identified for this gene. Rap1A is an important player in adhesion and migration of lymphocytes. It believed to interact with C-Raf, PDE6D, TSC2, RALGDS, RAPGEF2 and MLLT4.

Rap1A is also known in the art as KREV-1, KREV1, RAPT, and SMGP21. External IDs for Rap1A include: OMIM: 179520; MGI: 97852; HomoloGene: 2162; and GeneCards: RAP1A Gene. The human ortholog has been identified as follows: Entrez 5906; Ensembl ENSG00000116473; UniProt P62834; RefSeq (mRNA) NM_{—}001010935; RefSeq (protein) NP_{—}001010935; and Location (UCSC) Chr 1: 111.89-112.06 Mb. The mouse ortholog has been identified as follows: Entrez 109905; RefSeq (mRNA) NM_{—}145541; and RefSeq (protein) NP 663516.

### Cardiac Arrhythmia

Cardiac arrhythmia is a condition in which there is abnormal electrical activity in the heart. The heartbeat may be too fast or too slow, and may be regular or irregular. Arrhythmia may be classified by rate (normal, tachycardia, bradycardia), by mechanism (automaticity, reentry, fibrillation), and/or by site of origin. In particular, embodiments of the present invention are directed to ventricular arrhythmias, which include premature ventricular contractions (PVC), accelerated idioventricular rhythm, monomorphic ventricular tachycardia, polymorphic ventricular tachycardia, ventricular fibrillation, and long QT syndrome (LQTS). Another form of cardiac arrhythmia is proarrhythmia; a new or more frequent occurrence of pre-existing arrhythmias that can be precipitated by antiarrhythmic therapy. Proarrhythmia is a side effect associated with the administration of some antiarrhythmic drugs, as well as drugs for other indications.

Of particularly interest herein is the cardiac arrhythmia LQTS. Long QT syndrome is an inborn heart condition in which delayed repolarization of the heart following a heartbeat increases the risk of episodes of torsade de pointes, a form of irregular heartbeat that originates from the ventricles. These episodes may lead to palpitations, fainting and sudden death due to ventricular fibrillation. LQTS is so named because of the way it appears on an electrocardiogram, in which there is prolongation of the QT interval.

Approximately, 75% of patients with long QT have a known genetic cause, as discussed herein. The underlying genetic cause in the remaining 25% of patients remains unknown. As demonstrated herein, loss of Rap1A function may contribute to long QT in the human population. Therefore, testing Rap1A functional activity in bodily samples of patients can be used as a novel marker for diagnosis and for designing therapeutic strategies.

In one embodiment, the method is used to assess if a subject has cardiac arrhythmia, and in particular long QT syndrome. A subject has cardiac arrhythmia if they would be diagnosed by one skilled in the art of cardiovascular medicine has having cardiac arrhythmia on the basis of conventional symptoms, such as an abnormal awareness of heartbeat (i.e., palpitations), a heartbeat that is too fast, too slow or too weak that can manifest as lower blood pressure resulting in lightheadedness or dizziness, or ECG readings that indicate cardiac arrhythmia (e.g., long QT syndrome).

In another embodiment, the present methods are used to assess a subject's risk of developing (or having) cardiac arrhythmia (e.g., LQTS) in the future. In one embodiment, the subject at risk of developing cardiac arrhythmia is an apparently healthy individual. A subject at risk of developing cardiac arrhythmia has a higher than normal chance of exhibiting symptoms of cardiac arrhythmia at a later point in their lifetime. While low Rap1A activity predisposes a subject to eventually exhibit cardiac arrhythmia, it is difficult to determine when cardiac arrhythmia will actually manifest, as other factors such as the subjects activity level, general fitness, and other physical predispositions may influence if and when cardiac arrhythmia occurs. For example, a subject identified as being at risk of developing cardiac arrhythmia may develop cardiac arrhythmia within one year, within 5 years, within 10 years, within 20 years, or they may become deceased before developing cardiac arrhythmia. Nonetheless, it is useful to know if a subject is at risk of developing cardiac arrhythmia, as this can allow them to take steps to decrease the likelihood or consequences of their developing cardiac arrhythmia. For example, a subject diagnoses as being at risk of developing LQTS should avoid antiarrhythmic agents that could prolong the QT interval.

### Bodily Samples

Bodily samples include, but are not necessarily limited to bodily fluids such as blood-related samples (e.g., whole blood, serum, plasma, and other blood-derived samples), urine, cerebral spinal fluid, bronchoalveolar lavage, and the like. A preferred bodily sample is a peripheral blood sample that includes a blood cell selected from the group consisting of platelets, neutrophils, monocytes, and lymphocytes. Another example of a bodily sample is a tissue sample. For example, smooth muscle cells explanted from arterioles isolated from skin punch biopsy sample can be used as a tissue sample. Rap1A levels can be assessed either quantitatively or qualitatively, usually quantitatively. The levels of the Rap1A can be determined ex vivo.

A peripheral blood sample can be collected in the clinic, transported to the laboratory and processed to isolate and purify various blood cells, including platelets, neutrophils, monocytes, and lymphocytes. For example, lymphocytes cultured in the laboratory can be used to more accurately test for Rap1A functional activity by depriving cells of external stimuli present in vivo in human serum. Cells cultured under serum-free conditions can be stimulated with activators to determine the level of Rap1A functional activity. Low or impaired activity will implicate a defect in Rap1 function, classifying the patient at risk for arrhythmias.

A bodily sample may be fresh or stored (e.g. blood or blood fraction stored in a blood bank). The bodily sample may be a bodily fluid expressly obtained for the assays of this invention or a bodily fluid obtained for another purpose which can be subsampled for the assays of this invention.

Peripheral blood may be obtained from the subject using standard clinical procedures. The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions at physiological pH, such as phosphate, Tris, or the like, can be used.

Genomic DNA can be isolated from these cells to screen for mutations or deletions/duplications in the Rap1 gene. Mutations or single nucleotide polymorphisms (SNPs) that can impair Rap1 function include frameshift mutations, missense mutations affecting exon splice junctions or a critical amino acid necessary for biological activity. Currently, two SNPs of interest in Rap1A are known and include a missense mutation G changing glutamic acid to lysine, and a frameshift mutation that alters the peptide (UC Santa Cruz Human Genome Browser). None of these mutations have been linked to arrhythmias. New mutations may exist that can be identified by this approach. Similarly, blood cells can be used to examine expression levels of KCNH2, KCNQ1, and antisense transcript using qRT-PCR, and assay for Dnmt3a expression and activity. Alternatively, these assays can also be performed on smooth muscle cells explanted from arterioles isolated from skin punch biopsy sample of patients.

### Subjects

The subject is any human or other animal to be tested for characterizing its risk of cardiac arrhythmia. Subjects can also be referred to herein as patients. Subjects are generally a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In certain embodiments the subject is apparently healthy. “Apparently healthy”, as used herein, describes a subject who does not have any signs or symptoms of cardiac arrhythmia. Subjects having an elevated risk of cardiac arrhythmia include those with a family history of cardiac arrhythmia, or the presence of other markers for cardiac arrhythmia such as heart palpitations, irregular electrocardiogram (ECG) readings, or the presence of another marker such as a mutation in the genes KCNQ1, KCNH2, or SCN5A.

### Methods for Measuring the Activity of Rap1A

The activity of Rap1A can be evaluated using methods for determining the activity of proteins known by those skilled in the art. Because the activity of Rap1A can vary as a result of mutation of the Rap1A protein, it is important to measure the activity, and not just the level, or the Rap1A protein. In some embodiments, the Rap1A activity in the bodily sample is determined by Rap1 activation assay. Rap1 acts as a molecular on-off switch relaying extracellular signaling or information to intracellular target proteins by alternating between a GTP-bound active state and a GDP-bound inactive state. The Rap1 activation assay measures and quantitates Rap1 GTP binding. Rap1 activation is assessed by standard pull-down assays using cell lysates with Rap1-activation specific probe corresponding to 97 amino acids of human Ral-GDS-Rap-binding domain. Antibodies specific to Rap1 are used to analyze activated as well as total Rap1. This assay can also be performed in 96-well format for high-throughput screening of patient samples using a luminometer or spectrophotometer.

This assay essentially measures the biologically active form of Rap1 (Rap1-GTP bound form). This active form will recognize and bind to the 97 amino acids of human Ral-GDS-Rap-binding domain, whereas the inactive form (Rap1-GDP) will not. The 97 amino acid region can be pulled down or separated by sepharose beads. The bound Rap1 can be quantitated by Western blot analysis. The pull-down assay will also select another isoform of Rap1, the Rap1B isoform. However, Western blotting can be performed using anti-Rap1A specific antibody to distinguish between Rap1B and Rap1A. The data is usually normalized to the total Rap1A (both GTP and GDP bound forms) by Western blot analysis. The assay can be customized to a 96-well format for high-throughput screening.

The Rap activation assay is described in greater detail in Chotani et al., Am J Physiol. Heart Circ Physiol. [special “Translational Physiology” series article] 288: H69-H76; 2005 (PMID 15345481), the disclosure of which is incorporated by reference herein. As described by Chotani et al., Rap GTPase activation was assessed by Rap pull-down assays. Assays were performed on quiescent arteriolar vascular smooth muscle cells (VSM) that were treated with the cyclic AMP elevating agent forskolin (10 uM) for 0, 5, 10, 30 minutes. These assays were performed with 200 μg of cell lysate using the activation-specific probe corresponding to 97 amino acids of human Ral GDS-rap-binding domain (Upstate; Lake Placid, N.Y.). Total Rap protein in arteriolar VSM was also examined using 30 μg of the same cell lysate. Antibodies specific for Rap 1 (1:500 dilution for 1 h, at room temperature, Upstate), and Rap 2 (1:2,500 dilution for 1 h, at room temperature, BD Transduction Laboratories; Lexington, Ky.) were used to analyze activated as well as total Rap. Blots were developed by ECL Western blot detection reagents (Amersham Biosciences; Piscataway, N.J.) and quantitated by densitometry (Personal Densitometer, Molecular Dynamics; Sunnyvale, Calif.).” Forskolin activated Rap1 (maximum activation at 5 min), whereas Rap2 was unaffected by forskolin.

Once the levels of Rap1A have been determined, they can be displayed in a variety of ways. For example, the levels of choline-related trimethylamine-containing compounds can be displayed graphically on a display as numeric values or proportional bars (i.e., a bar graph) or any other display method known to those skilled in the art. The graphic display can provide a visual representation of the amount of the Rap1A in the bodily sample being evaluated. In addition, in some embodiments, the analytic device can also be configured to display a comparison of the levels of Rap1A in the subject's bodily fluid to a control value based on levels of Rap1A in comparable bodily fluids from a reference cohort.

In additional embodiments, subjects may also be evaluated for other markers for cardiac arrhythmia. For example, an increased level of Dnmt3a is also associated with the occurrence of cardiac arrhythmia. Dnmt3a is a DNA methytransferase that methylates the pyrimidine base cytosine giving 5-methylcytosine. In some embodiments the level of Dnmt3a in the bodily sample is determined by Western analysis. The biological activity of Dnmt3a in bodily samples can also be determined by Dnmt3a direct activity assay. Incubation of bodily sample with S-adenosylmethionine and Dnmt3a substrate in a 96-well format can be used to determine Dnmt3a biological activity for high throughput screening of patient samples using a luminometer or fluorescent microplate reader.

Comparison of a Rap1A Obtained from a Subject to a Control Value

In certain embodiments, the activity of Rap1A protein in a bodily sample of the subject is compared to the activity of Rap1A protein from the bodily sample of the subject to at least one Rap1A control. Subjects exhibiting a decreased level of Rap1A activity in the bodily sample as compared to the at least one Rap1A control has or is at risk of developing cardiac arrhythmia (e.g., LQTS). Moreover, the extent of the difference between the subject's Rap1A activity and the control value is also useful for characterizing the extent of the risk and thereby, determining which subjects would most benefit from certain therapies.

In certain embodiments, levels of Rap1A in the bodily sample obtained from the subject may be compared to a control value. A control value is a number representing the level of activity of Rap1A corresponding to that seen in one or more healthy subjects. For example, the control value can be based upon the activity of Rap1A in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of cardiac arrhythmia. In certain embodiments, the reference cohort includes individuals, who if examined by a medical professional would be characterized as free of symptoms of cardiac arrhythmia (e.g., LQTS). Appropriate categories can be selected with no more than routine experimentation by those of ordinary skill in the art.

The control value is preferably measured using the same units used to characterize the activity of Rap1A obtained from the subject. Thus, if the level of the Rap1A is an absolute value such as the activity of Rap1A per ml of blood, the control value is also based upon the activity of Rap1A per ml of blood in individuals in the general population or a select population of human subjects.

As noted herein, the extent of the difference between the subject's Rap1A activity and the control value is useful for characterizing the extent of the risk that the subject will develop cardiac arrhythmia. Depending on the difference in activity seen between the subject and the control, the subject may be categorized as belonging to various risk categories, such as a low risk group, a medium risk group and a high-risk group, with the test subject's risk of having cardiac arrhythmia can be based upon which group his or her test value falls. Control values of Rap1A in bodily samples obtained, such as mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each risk predictor that is assayed.

The activity level of Rap1A in a subject's bodily sample may be compared to a single control value or to a range of control values. If the level of Rap1A in the subject's bodily sample is less than the control value, the subject is at greater risk of currently having or developing cardiac arrhythmia or experiencing an adverse cardiac event within the ensuing year, two years, and/or three years than individuals with levels comparable to or below the control value or in the lower range of control values. In contrast, if the activity of Rap1A in the test subject's bodily sample is above or equivalent to the control value, the test subject is at a lower risk of developing or having cardiac arrhythmia. In those cases where the difference is being used to categorize the level of risk, such as the activity ranges for individuals at high risk, average risk, and low risk, the comparison involves determining into which group the test subject's activity level of Rap1A falls.

In another embodiment, the present invention relates to kits that include reagents for assessing activity of Rap1A in bodily samples obtained from a subject. In certain embodiments, the kits also include printed materials such as instructions for practicing the present methods, or information useful for assessing a subject's risk of cardiac arrhythmia. Examples of such information include, but are not limited to cut-off values, sensitivities at particular cut-off values, as well as other printed material for characterizing risk based upon the outcome of the assay. In some embodiments, such kits may also comprise control reagents, e.g. an amount of Rap1A having a known activity.

### Administration of Antiarrhythmic Agents

Embodiments of the methods described herein can also be useful for determining if and when antiarrhythmic agents for treating cardiac arrhythmia should or should not be prescribed for an individual. For example, individuals with Rap1A values above a certain cutoff value, or that are in the higher tertile or quartile of a “normal range,” could be identified as those in need of more aggressive intervention with antiarrhythmic agents, life style changes, etc.

The method for assessing if a subject has or is at risk of developing cardiac arrhythmia can further include the step of administering a therapeutically effective amount of an antiarrhythmic agent to a subject that has or is at risk of developing cardiac arrhythmia. This is particularly advised if the subject has cardiac arrhythmia. Administration of a therapeutically effective amount of an antiarrhythmic agent can reduce the symptoms and/or protect the subject from the effects of cardiac arrhythmia.

Antiarrhythmic agents are a group of pharmaceuticals used to suppress abnormal rhythms of the heart (i.e., cardiac arrhythmias). Antiarrhythmic agents are organized into five different classes, with classes I through IV having mechanisms of action that affect sodium channels, β1-adrenergic receptors, potassium channels, and calcium channels, respectively. Preferred antiarrhythmic agents for use in treating cardiac arrhythmias identified using the methods described herein include Class I antiarrhythmics and class II antiarrhythmics. Class I antiarrhythmics include, for example, the agents quinidine, procainamide, disopyramide, lidocaine, phenytoin, mexiletine, tocainide, flecainide, propafenone, and moricizine. Class II antiarrhythmics include, for example, the agents propranolol, esmolol, timolol, metoprolol, atenolol, and bisoprolol.

The term “therapeutically effective” is intended to qualify the amount of antiarrhythmic agent which will achieve the goal of reducing disease severity and the frequency of incidence of the symptoms of cardiac arrhythmia over the period of treatment, while avoiding adverse side effects. The dosage form and amount can be readily established by reference to known treatment or prophylactic regiments, or can be determined using appropriate animal models.

The amount of therapeutically active compound that is administered and the dosage regimen for treating cardiac arrhythmia depends on a variety of factors, including the age, weight, sex, and medical condition of the subject, the severity of the disease, the route and frequency of administration, and the particular compound employed, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of antiarrhythmic agent to be administrated may need to be optimized for each individual. The pharmaceutical compositions may contain active ingredient in the range of about 0.1 to 500 mg, preferably in the range of about 0.5 to 200 mg and most preferably between about 1 and 100 mg. A daily dose of about 0.01 to 50 mg/kg body weight, preferably between about 0.1 and about 20 mg/kg body weight, may be appropriate. The daily dose can be administered in one to four doses per day.

The antiarrhythmic agent may be administered in any method, and suitable method for administration may be determined in accordance with various forms of preparation, ages, sexes and other conditions of the subject, the degree of severity of disease, and the like. For example, tablets, pills, solutions, suspensions, emulsion, granules and capsules are administered orally. The injections are intravenously administered alone or together with a conventional auxiliary liquid (e.g. glucose, amino acid solutions), and further are optionally administered alone in intramuscular, intracutaneous, subcutaneous, or intraperitoneal route, if desired. Suppositories are administered in intrarectal route.

Subjects diagnosed with long QT syndrome are usually advised to avoid drugs that would prolong the QT interval further or lower the threshold for TDP. There are two main options for intervention for individuals with LQTS: arrhythmia prevention and arrhythmia termination.

Arrhythmia prevention involves the use of medications or surgical procedures that attack the underlying cause of the arrhythmias associated with LQTS. Since the cause of arrhythmias in LQTS is after depolarization, and these depolarizations are increased in states of adrenergic stimulation, steps can be taken to blunt adrenergic stimulation in these individuals. These include: administration of beta receptor blocking agents which decreases the risk of stress induced arrhythmias; potassium supplementation since if potassium content in the blood rises, the action potential shortens and it is believed that increasing potassium concentration could minimize the occurrence of arrhythmias; administration of a sodium channel blocker such as mexiletine; and amputation of the cervical sympathetic chain (left stellectomy).

Cardiac arrhythmia, as defined herein, includes subjects who are currently suffering from irregular heart rhythm. However, this arrhythmia is in many cases not immediately life threatening. Arrhythmia termination is a method of restoring normal cardiac rhythm. Life-threatening arrhythmia is a subset of cardiac arrhythmia, and must be treated immediately or the subject will die. One effective form of arrhythmia termination in individuals with LQTS is placement of an implantable cardioverter-defibrillator (ICD). Alternatively, external defibrillation can be used to restore sinus rhythm. ICDs are commonly used in patients with syncopes despite beta blocker therapy, and in patients who have experienced a cardiac arrest.

### Evaluation of Antiarrhythmic Agents

Another aspect of the invention provides a method of evaluating the efficacy of treatment of a subject having decreased Rap1A activity with an antiarrhythmic agent. Examples of suitable antiarrhythmic agents include class I antiarrhythmic agents and class II antiarrhythmic agents. The method includes identifying a subject having decreased Rap1A activity, administering a therapeutically effective amount of an antiarrhythmic agent to the subject, determining the level of a symptom of cardiac arrhythmia in the subject; comparing the level of the symptom of cardiac arrhythmia to a corresponding predetermined value, and determining the treatment to be efficacious if the activity of the level of the symptom of cardiac arrhythmia has decreased in comparison to the predetermined value. As described herein, a variety of symptoms of cardiac arrhythmia are known, including increased sodium channel activity, prolongation of the QT interval, palpitations, dizziness, fainting, and shortness of breath.

Evaluation of the efficacy of antiarrhythmic agents can include obtaining a predetermined value for a symptom of cardiac arrhythmia, and determining the level of the symptom in a subject following administration of the therapeutic agent. A decrease in the symptom of cardiac arrhythmia after administration of the antiarrhythmic agent as compared to the level of the symptom of cardiac arrhythmia obtained before administration of the antiarrhythmic agent is indicative of a positive effect of the antiarrhythmic agent on cardiac arrhythmia in the treated subject.

A predetermined value can be based on an evaluation of the symptom of cardiac arrhythmia in the subject prior to administration of an antiarrhythmic agent. In another embodiment, the predetermined value is based on the level of the symptom of cardiac arrhythmia seen in control subjects that have been identified as having low levels of Rap1A activity. The predetermined value will correspond to whatever symptom is currently being evaluated in the method, so that, for example, if palpitations are measured, the predetermined value will be a measure of palpitations prior to administration of the antiarrhythymic agent.

Based on the understanding of Rap1A function described herein, activation of Rap1A leads to inactivation of the sodium channel. Furthermore, in the absence of Rap1A signaling, increased sodium channel activity can occur. Accordingly, if treatment of a subject having been identified as having low Rap1A activity with an antiarrhythmic agent is effective, the sodium channel activity should decrease from what was initially observed in the subject; i.e., the predetermined value.

Accordingly, in one embodiment, evaluation of the symptom of cardiac arrhythmia involves determining the level of cardiovascular sodium channel activity in a cardiovascular sample taken from the subject following administration of the therapeutic agent and comparing it to a corresponding predetermined value. A decrease in the cardiovascular sodium channel activity in the sample taken after administration of the antiarrhythmic agent as compared to the predetermined value taken before administration of the antiarrhythmic agent is indicative of a positive effect of the antiarrhythmic agent on cardiac arrhythmia in the treated subject. For example, sodium channel activity can be evaluated by whole-cell patch-clamping technique on isolated cells.

In another embodiment, the symptom of cardiac arrhythmia is prolongation of the QT interval. This symptom can be particularly significant if the cardiac arrhythmia is long QT syndrome. In this embodiment, evaluation of the symptom of cardiac arrhythmia involves determining the level of QT prolongation seen in an ECG reading obtained from the subject following administration of the therapeutic agent and comparing it to a corresponding predetermined value. A decrease in the QT prolongation after administration of the antiarrhythmic agent as compared to the predetermined value for QT prolongation taken before administration of the antiarrhythmic agent is indicative of a positive effect of the antiarrhythmic agent on cardiac arrhythmia in the treated subject.

In another embodiment, the symptom of cardiac arrhythmia is palpitations. Palpitations are felt by the subject as an irregular or rapid pulsing of the heart. Examples of palpitations include skipped heartbeats, fluttering heartbeats, heartbeats that are too fast, and heartbeats that involve pumping that is harder than usual. Heart palpitations may be felt in the throat or neck, as well as the chest, and can occur whether the subject is active or at rest. In particular, the symptom of cardiac arrhythmia can be frequent and repeated palpitations.

In this embodiment, evaluation of the symptom of cardiac arrhythmia involves determining the level of palpitations reported by the subject following administration of the therapeutic agent and comparing it to a corresponding predetermined value. A decrease in the palpitations after administration of the antiarrhythmic agent as compared to the predetermined value for QT prolongation taken before administration of the antiarrhythmic agent is indicative of a positive effect of the antiarrhythmic agent on cardiac arrhythmia in the treated subject.

### Studies of Rap1A in Rap1A-Deficient Mice

Studies of the significance of Rap1A in cardiac arrhythmia have been carried out in Rap1A-deficient (knockout) mice. The genetically modified Rap1A-deficient (knockout) mouse model was originally developed by Dr. Lawrence A. Quilliam at the Indiana University School of Medicine, Department of Biochemistry and Molecular Biology. Breeding pairs of Rap1A knockout mice were kindly provided by Dr. Quilliam for cardiovascular studies on α_{2C}-adrenoceptors. Initially, Rap1A knockout mice were viable, healthy, and fertile, suggesting that this GTPase was dispensable for breeding, development, and behavior necessary for a normal life span. However, when these mice were backcrossed for six generations or more into a C57BL/6J mouse strain to obtain a congenic background, cardiac defects were more apparent.

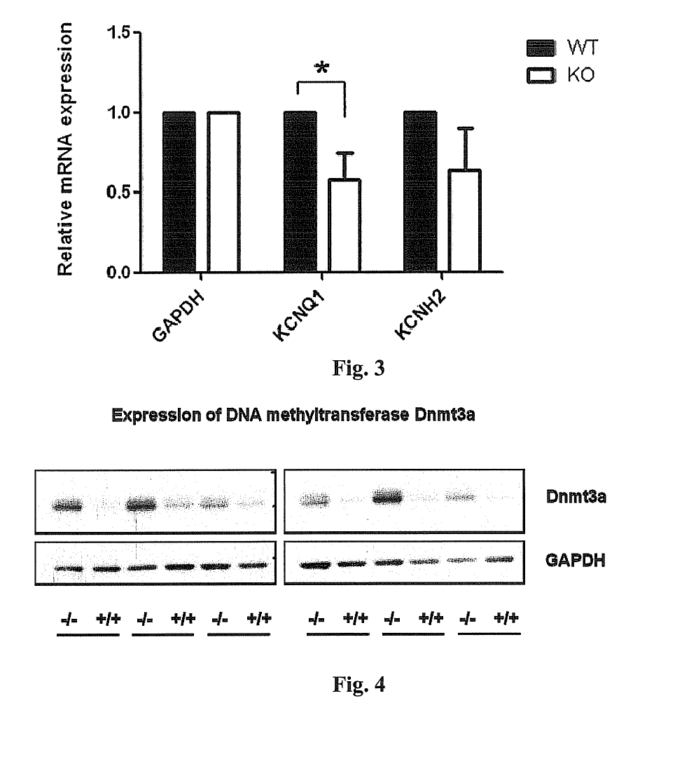

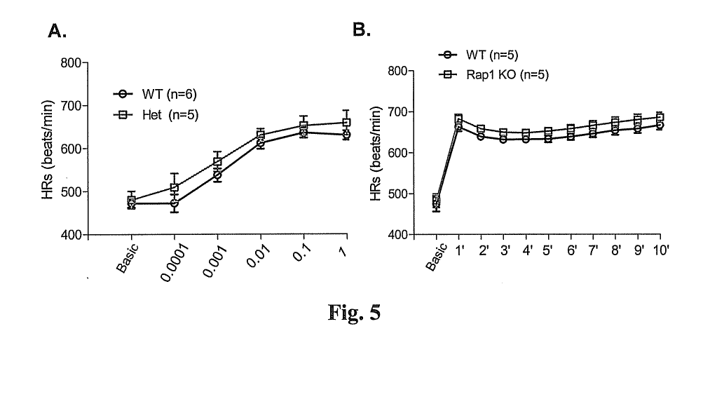

The in vivo cardiovascular status of Rap1A-deficient mice (Rap1A^{-/-}) and wild-type (WT) controls was measured by echocardiography and ECG (n=5/group). See FIG. 2. At rest, no significant difference in heart rate, left ventricle (LV) fractional shortening, or cardiac output, but a slight reduction in systolic blood pressure were observed in the two groups. In contrast, significant increases in QT interval (41.4±4.1 ms for WT and 57.6±2.5 ms for Rap1A^{-/-}; p<0.05), QRS duration (11.9±1.0 ms for WT and 15.9±1.4 ms for Rap1A^{-/-}; p<0.05), and R-R variability were observed in Rap1A^{-/-} mice. Evaluation of the ERG1 and KCNQ1, long QT associated voltage-activated potassium channels, with qRT-PCR and Western blotting indicate a 2-fold decrease in the mRNA and protein levels, as shown in FIG. 3. Studies have also determined an increase in the DNA methyltransferase, Dnmt3a, in Rap1A^{-/-} mice. See FIG. 4. MicroRNA (miRNA) array data indicates an up-regulation of the miR-467 cluster in Rap1A knockout heart tissue. Several members of this cluster have potential binding sites in the 3′-UTR of KCNQ1 mRNA. These results suggest mechanisms of transcriptional and post-transcriptional regulation for this potassium channel. Genetic ablation of Rap1A shows an impact on critical repolarization mechanisms. Although the two Rap1 subtypes A and B are closely related, they are not functionally redundant, with Rap1A contributing to normal cardiac function. The determination that the Rap1A^{-/-} presents a clinical long QT syndrome and that the heart tissue of these mice have altered potassium channel expression, provides a unique system to study the regulation of these ion channels. These findings indicate a novel genetic basis of prolonged QT interval and arrhythmia risks in vivo.

The present invention is illustrated by the following example. It is to be understood that the particular examples, materials, amounts, and procedures are to be interpreted broadly in accordance with the scope and spirit of the invention as set forth herein.

### Example

### Electrophysiological Abnormalities in Mice with Genetic Ablation of Rap1A GTPase

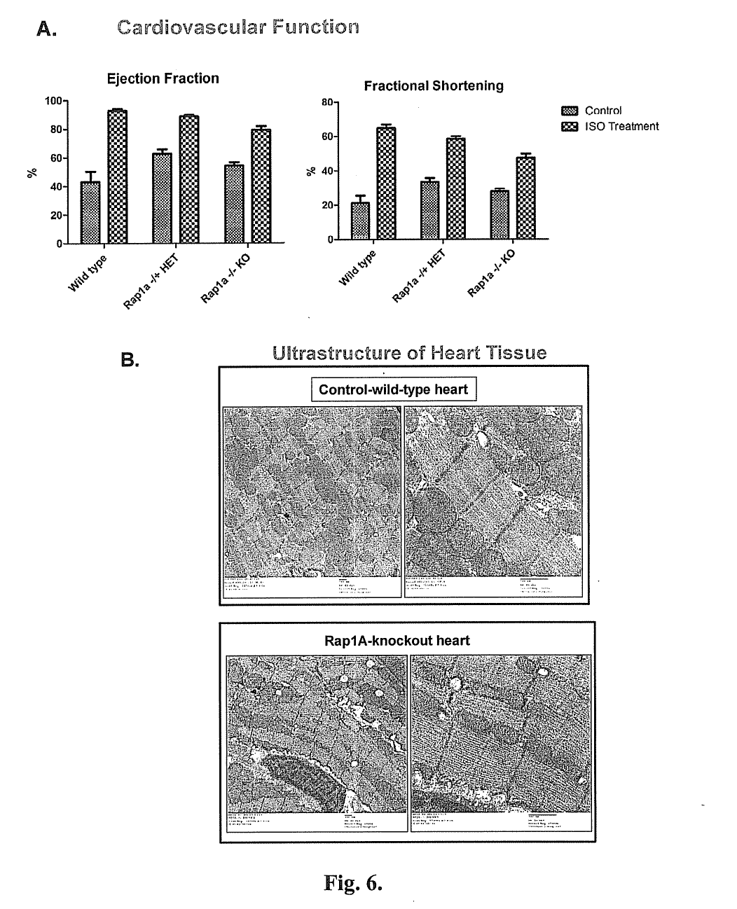

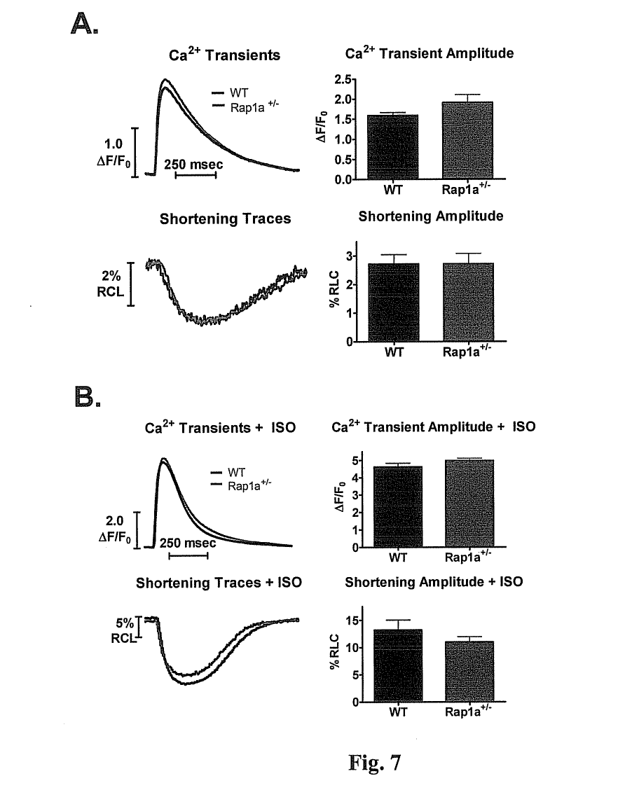

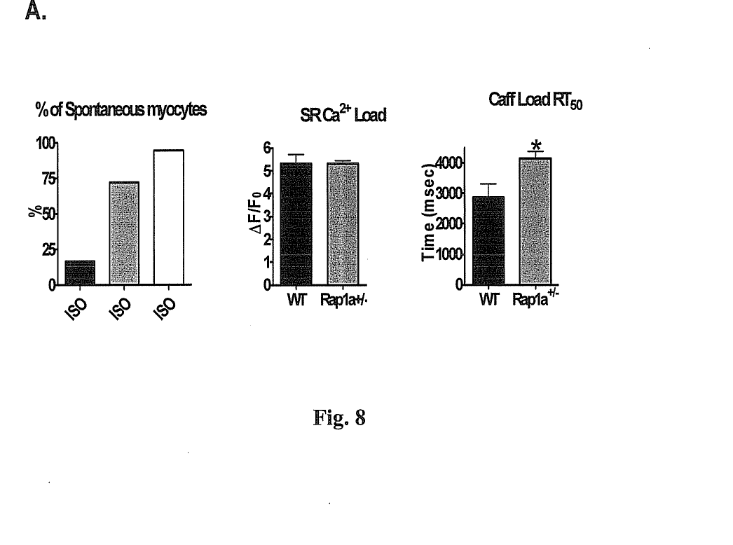

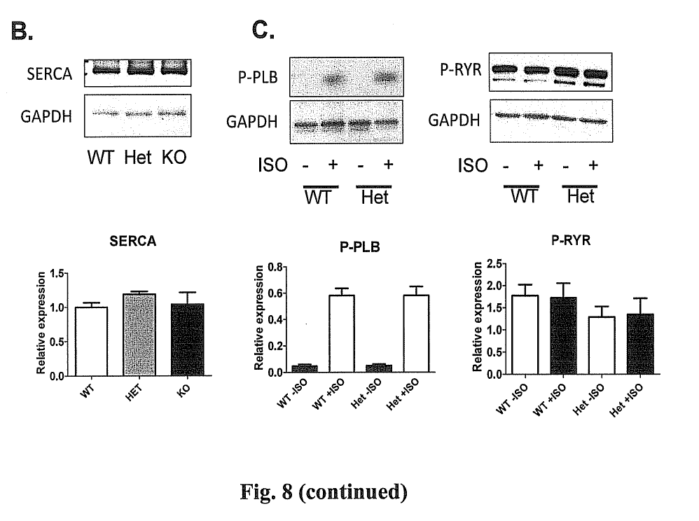

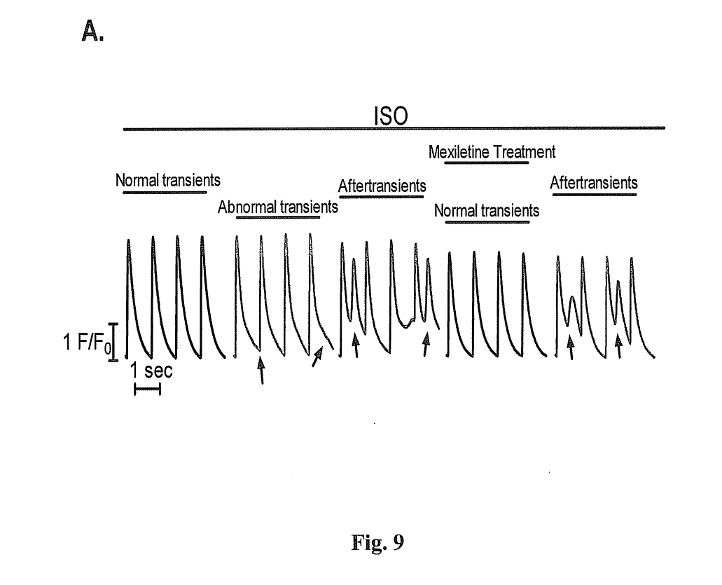

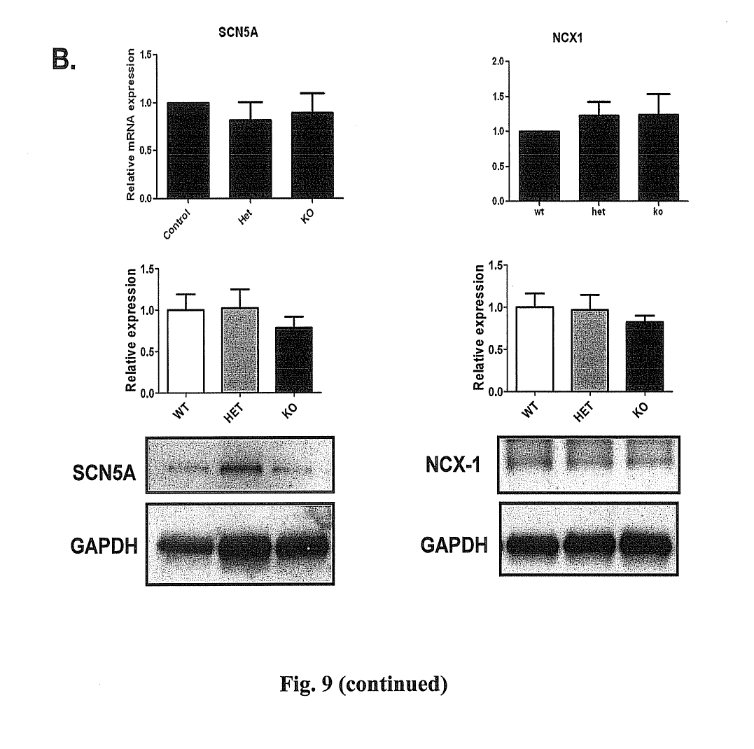

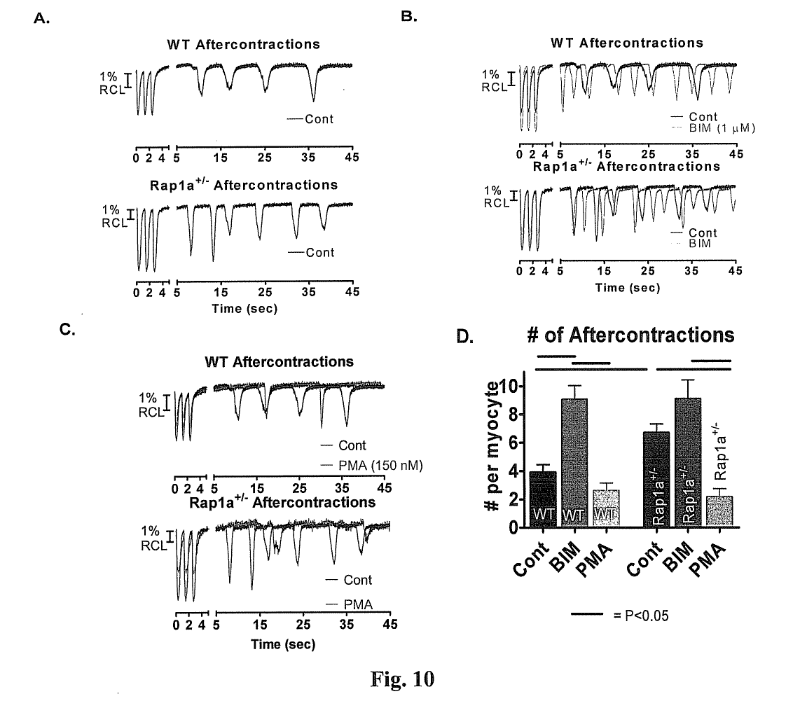

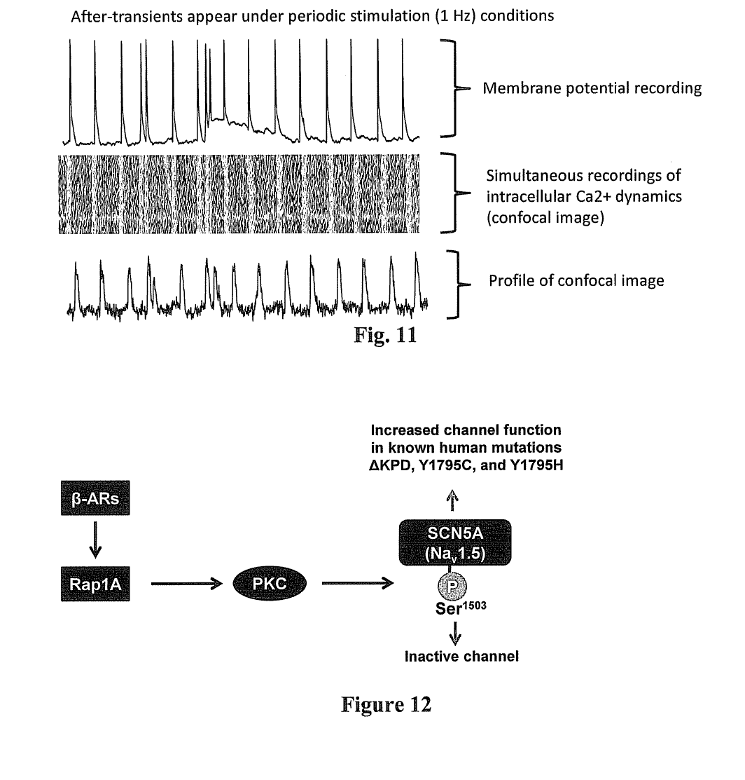

Rap1A, a member of the Ras-like GTPase family, acts as a molecular switch coupling extracellular stimulation to intracellular signaling through cyclic AMP. The impact of Rap1 deficiency in heart function had not heretofore been defined. The hypothesis that Rap1a null mice (Rap1A−/−) have abnormal ventricular excitation or contraction was tested, using in vivo cardiovascular status measures by echocardiography, and ECGs (n=5/group) in comparison to wild-type (WT) controls. The results are shown in FIGS. 5-11. Isoproterenol was used to activate the beta-adrenergic receptors which are known to increase intracellular cyclic AMP and to activate A-kinase and Rap1A signaling in the heart.

At rest, no significant difference in heart rate, LV fractional shortening, or cardiac output, but slight reduction in systolic blood pressure were observed in the two groups. In contrast, significant increases in QT interval (41.4±4.1 ms for WT and 57.6±2.5 ms for Rap1A−/−; p<0.05), QRS duration (11.9±1.0 ms for WT and 15.9±1.4 ms for Rap1A−/−; p<0.05), and R-R variability were observed in Rap1A−/− mice. Evaluation of the ERG1, long QT associated voltage-activated potassium channel, with qRT-PCR and Western blotting indicate a 2-fold decrease in the mRNA and protein levels. Together, loss of Rap1A shows an impact on repolarizing mechanisms. Although the two Rap1 subtypes A and B are closely related, they are not functionally redundant, with Rap1A contributing to normal cardiac function.

These findings suggest a novel genetic basis of prolonged QT interval and arrhythmia risks in vivo. More specifically, the studies demonstrate that Rap1A function is necessary for sodium channel physiological activity in the heart, and that loss of Rap1A function can lead to increased sodium channel activity, similar to the increased channel activity seen in known human mutations. A working model explaining the anti-arrhythmic effect of beta-adrenergic activation on Rap1A and sodium channel SCN5A is shown in FIG. 12.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing operation of the invention, the inventors are not bound by theories described herein. The invention is not limited to the exact details shown and described, for variations obvious to one skilled in the art will be included within the invention defined by the claims.

What is claimed is:

1. A method for assessing if a subject has or is at risk of developing cardiac arrhythmia, comprising:
a) determining the activity of Rap1A protein in a bodily sample of the subject; and
b) comparing the activity of Rap1A protein from the bodily sample of the subject to at least one Rap1A control,
wherein a decreased level of Rap1A activity in the bodily sample of the subject as compared to the at least one Rap1A control indicates that the subject is at risk of developing or has cardiac arrhythmia.

2. The method of claim 1, wherein the cardiac arrhythmia is Long QT syndrome.

3. The method of claim 1, further comprising the step of administering a therapeutically effective amount of an antiarrhythmic agent to a subject that has or is at risk of developing cardiac arrhythmia.

4. The method of claim 3, wherein the antiarrythmic agent is a class I antiarrhythmic agent.

5. The method of claim 3, wherein the antiarrhythmic agent is a class II antiarrhythmic agent.

6. The method of claim 1, wherein the bodily sample is a peripheral blood sample that includes a blood cell selected from the group consisting of platelets, neutrophils, monocytes, and lymphocytes.

7. The method of claim 1, wherein the Rap1A activity in the bodily sample is determined by Rap1 activation assay.

8. The method of claim 1, wherein the subject is human.

9. A method of evaluating the efficacy of treatment of a subject having decreased Rap1A activity with an antiarrhythmic agent, comprising:
a) identifying a subject having decreased Rap1A activity,
b) administering a therapeutically effective amount of an antiarrhythmic agent to the subject,
c) determining the level of a symptom of cardiac arrhythmia in the subject;
d) comparing the level of the symptom of cardiac arrhythmia to a corresponding predetermined value, and
determining the treatment to be efficacious if the level of the symptom of cardiac arrhythmia has decreased in comparison to the predetermined value.

10. The method of claim 9, wherein the symptom of cardiac arrhythmia is increased cardiovascular sodium channel activity.

11. The method of claim 9, wherein the symptom of cardiac arrhythmia is prolongation of the QT interval.

12. The method of claim 9, wherein the symptom of cardiac arrhythmia is palpitations.

13. The method of claim 9, wherein the antiarrhythmic agent is a class I antiarrhythmic agent.

14. The method of claim 9, wherein the antiarrhythmic agent is a class II antiarrhythmic agent.

15. The method of claim 9, wherein the subject is human.
